(19) United States
(12) Patent Application Publication
(10) Pub. No.: US 2009/0264931 A1
(43) Pub. Date: Oct. 22, 2009

(54) Implantable Article for Use with an Anchor and a Non-Metal Rod

(75) Inventors: Keith E. Miller, Memphis, TN (US); Lauren I. Lyons, Memphis, TN (US); Harold Taylor, Memphis, TN (US); Christopher M. Patterson, Memphis, TN (US); Dimiti K. Protopsaltis, Memphis, TN (US); William A. Rezach, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/105,552

(22) Filed: Apr. 18, 2008

(51) Int. Cl. A61B 17/70

(52) U.S. Cl. 606/251; 606/250

(57) ABSTRACT

An implantable article for use with an anchor and a non-metal rod assembly includes a ring having a body including an outer surface, an inner surface defining an aperture extending through the body configured to engage a non-metal rod. The ring body further includes protrusions extending from the inner surface into the aperture, wherein the protrusions are spaced apart from each other axially and circumferentially along the inner surface.

### FIELD OF THE DISCLOSURE

This disclosure is directed to an implantable article, and more particularly directed toward an implantable ring for use with a clamp assembly for connecting an anchor and a non-metal rod.

### DESCRIPTION OF THE RELATED ART

There are a variety of spinal diseases, such as scoliosis, which may be cured or mitigated by implantation of certain devices. For example, in patients with scoliosis, an anchor and rod implant assembly may be used to change an improper curvature by aligning the spine with the rod via anchors or hooks. Generally, for such implants, the anchors are attached to the spine, for example, screws driven into particular locations within the spine, which are also affixed to rods that provide a rigid support for adjusting the spinal deformity. Typically, a number of screws can be inserted within the spine, for example in the pedicles, and the rod can be attached to the screws such that the spine is encouraged to reform itself and align itself with the rod.

As surgeons develop and invent new ways to treat certain spinal deformities, the number of implants and the types of implants for correcting such deformities increases. However, because of the nature of treating spinal deformities, and the critical function of the spine, such implants and methods of treating deformities must be suitably developed to ensure patient recovery and proper implant performance. Accordingly, the industry continues to demand improvements in implants including implants that are safer, have longer lasting lifetimes, and give surgeons greater options in treatment methods.

### BRIEF DESCRIPTION OF THE DRAWINGS

The present disclosure may be better understood, and its numerous features and advantages made apparent to those skilled in the art by referencing the accompanying drawings.

FIG. 1 includes a lateral view of a portion of a vertebral column.

FIG. 2 includes a top plan view of a vertebrae.

FIG. 3 includes a perspective view of a clamp assembly including a ring and a portion of an anchor in accordance with one embodiment.

FIG. 4 includes a perspective view of a ring configured for use in a clamp assembly in accordance with one embodiment.

FIG. 5A includes a cross-sectional view of the ring of FIG. 4 in accordance with one embodiment.

FIG. 5B includes a cross-sectional view of the ring of FIG. 4 in accordance with one embodiment.

FIG. 6A includes a perspective view of a ring configured for use in a clamp assembly in accordance with one embodiment.

FIG. 6B includes a perspective view of a ring configured for use in a clamp assembly in accordance with one embodiment.

FIG. 6C includes a cross-sectional view of the ring of FIG. 6B configured for use in a clamp assembly in accordance with one embodiment.

FIG. 6D includes a perspective view of a ring configured for use in a clamp assembly in accordance with one embodiment.

FIG. 6E includes a cross-sectional view of the ring of FIG. 6D configured for use in a clamp assembly in accordance with one embodiment.

FIG. 7 includes a perspective view of a ring configured for use in a clamp assembly in accordance with one embodiment.

FIG. 8 includes a perspective view of a ring configured for use in a clamp assembly in accordance with one embodiment.

FIG. 9 includes a cross-sectional illustration of the ring of FIG. 8 in accordance with one embodiment.

FIG. 10 includes a perspective view of a ring configured for use in a clamp assembly in accordance with one embodiment.

FIG. 11 includes a cross-sectional illustration of the ring of FIG. 10 in accordance with one embodiment.

FIG. 12 includes a perspective view of a ring configured for use in a clamp assembly in accordance with one embodiment.

FIG. 13 includes an illustration of the ring of FIG. 12 in accordance with one embodiment.

FIG. 14 includes a perspective view of a ring configured for use in a clamp assembly in accordance with one embodiment.

FIG. 15 includes a perspective view of a ring configured for use in a clamp assembly in accordance with one embodiment.

FIG. 16 includes a perspective view of a ring having multiple portions and configured for use in a clamp assembly in accordance with one embodiment.

FIG. 17 includes an illustration of the ring of FIG. 16 after assembly in accordance with one embodiment.

FIG. 18 includes a perspective view of a ring having multiple portions and configured for use in a clamp assembly in accordance with one embodiment.

FIG. 19 includes an illustration of the ring of FIG. 18 after assembly in accordance with one embodiment.

FIG. 20 includes a perspective view of a ring having multiple portions and configured for use in a clamp assembly in accordance with one embodiment.

FIG. 21 includes a cross-sectional view of the ring of FIG. 20 configured for use in a clamp assembly in accordance with one embodiment.

The use of the same reference symbols in different drawings indicates similar or identical items.

### DETAILED DESCRIPTION

### Description of Relevant Anatomy

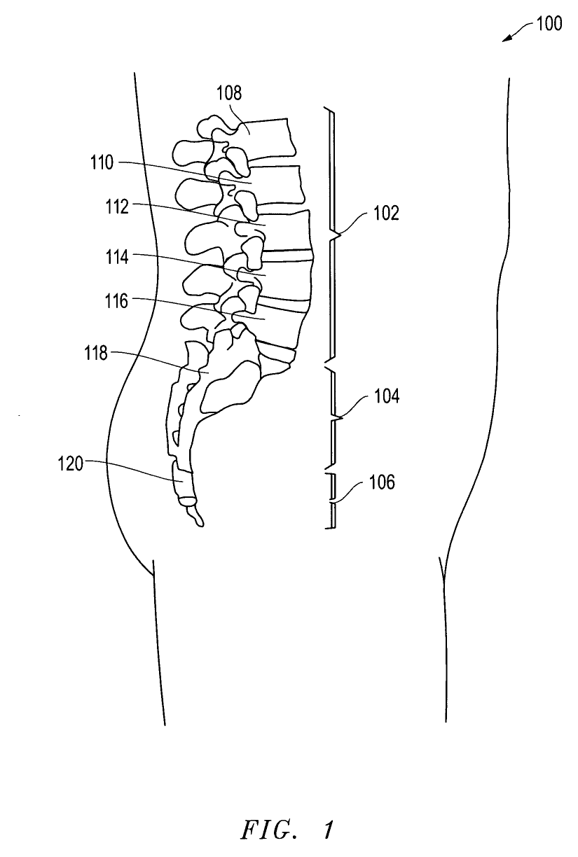

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

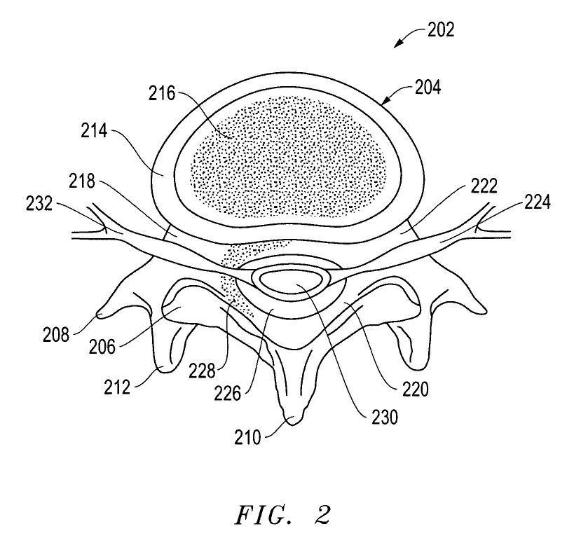

Referring to FIG. 2, a vertebra 202 is illustrated. As shown, the vertebral body 204 includes a cortical rim 214 composed of cortical bone. Also, the vertebral body 204 includes cancellous bone 216 within the cortical rim 214. The cortical rim 214 is often referred to as the apophyseal rim or apophyseal ring. Further, the cancellous bone 216 is generally softer than the cortical bone of the cortical rim 214.

As illustrated in FIG. 2, the vertebra 202 further includes a first pedicle 222, a second pedicle 218, a first lamina 220, and a second lamina 228. Further, a vertebral foramen 226 is established within the vertebra 202. A spinal cord 230 passes through the vertebral foramen 226. Moreover, a first nerve root 224 and a second nerve root 232 extend from the spinal cord 230. Notably, during implantation of anchors, such as screws within the spine, particularly anchors that will be attached to other implants, such as a rod, such screws can generally be implanted within the pedicles 218 and 222, since these portions of the spine provide suitable support and rigidity for anchors.

The vertebrae that make up the vertebral column have slightly different appearances as they range from the cervical region to the lumbar region of the vertebral column. However, all of the vertebrae, except the first and second cervical vertebrae, have the same basic structures.

### DESCRIPTION OF EMBODIMENTS OF THE IMPLANTABLE ARTICLE

In some instances, it is the preference of the surgeon to use a non-metal rod as an implant to avoid stress-shielding effects. Generally, stress-shielding effects can result in a patient lacking sufficient bone density, as the implant shields the surrounding bone from environmental stresses that would otherwise result in strengthening (i.e., densification) of the bone, and as a result the patient is left with less than desirable bone density around an implant. Accordingly, surgeons may opt to use less rigid implant assemblies to reduce the stress-shielding effects for some patients in order for their body to recover to a stronger state. One particular example includes the use of non-metal rods for rod and anchor assemblies in correcting spinal deformities. However, non-metal rods may be more susceptible to fatigue and fracture when coupled with more rigid, metal components, as the contact region with the metal component can create a region of localized stress in the non-metal rod, leading to fracture of the non-metal rod. The implantable articles described herein are particularly suited for use with non-metal rod assemblies.

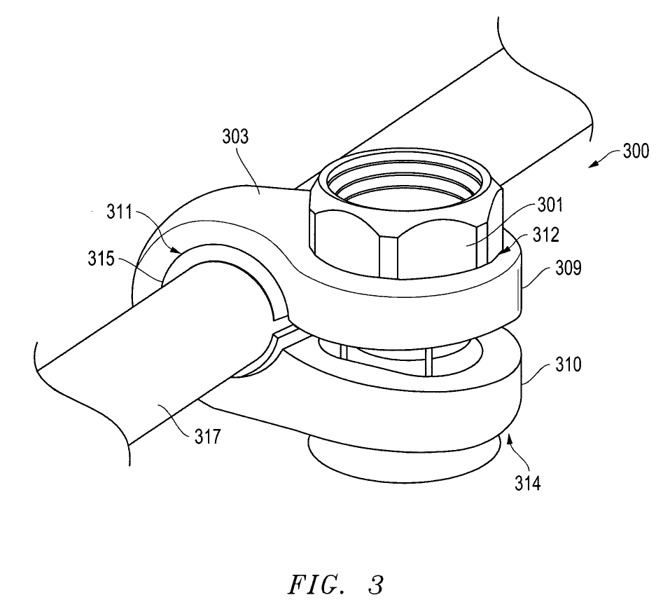

Referring to FIG. 3, a perspective view of a clamp assembly 300 is illustrated in accordance with one embodiment. Typically, such clamp assemblies are suitable for joining a rod with an anchor implanted within the human body. The clamp assembly 300 includes a clamp 303 having arms 309 and 310. Typically, each of the arms 309 and 310 have openings 312 and 314 configured to engage an anchor portion 301 that can extend through the openings 312 and 314. The clamp 303 can further include an opening 311 adjacent to the arms 309 and 310 configured to engage a ring 303 therein. In one embodiment, the ring is rotateably coupled within the opening 311 of the clamp 303 such that it can articulate within the opening 311 before tightening, such that a proper orientation between the rod 317, clamp 303, and anchor portion 301 is obtained.

A rod 317 can be engaged by the clamp 303 within the opening 311 and more particularly by extension through the ring 303 disposed within the opening 311. The ring 305 can be fitted onto the rod 317, and the ring 305 can be disposed within the opening 311 of the clamp 303. The clamp assembly 300 can then combine the rod 317 and anchors, such that upon tightening of the anchor portion 310, the arms 309 and 310 of the clamp 303 are compressed towards each other, compressing the opening 311 and the ring 315, thereby fixably engaging the rod 317 therein. Such a design fixes the position of the anchor relative to the rod 317. Typically, the clamp 303 and the anchor portion 301 can include a metal or metal alloy. According to one embodiment, suitable metals can include cobalt, chromium, tungsten, nickel, cobalt, titanium, molybdenum, and any combination thereof.

FIG. 4 includes a perspective view of a ring configured to be used within a clamp assembly in accordance with one embodiment. The ring 400 includes an outer surface 401, an inner surface 403 that defines an aperture 408 extending through the body of the ring along the axial direction 407. The ring 400 further includes protrusions 404, 405, and 406 (404-406) extending from the inner surface 403 into the aperture 408, and configured to engage a non-metal rod extending through the aperture 408 along the axial direction 407. According to one embodiment, the protrusions 404-406 are placed along the inner surface 403 such that they are displaced from each other in an axial direction and circumferentially spaced apart. As illustrated in FIG. 4, the axial direction 407 is a direction extending through the aperture 408 and a radial direction 409 is a direction perpendicular to the axial direction 407 extending from the center of the aperture 408 through the inner surface 403 and outer surface 401 of the ring 400. Moreover, a circumferential direction 417 extends along the circumference of the surfaces (e.g., the outer surface 401 or inner surface 403) of the ring body 400. As illustrated the protrusions 404-406 are axially and circumferentially spaced apart.

As illustrated, and in accordance with one embodiment, the protrusions 404-406 can be discrete hemispherical projections axially spaced apart and circumferentially spaced apart along the inner surface. In another embodiment, the discrete hemispherical projections 404-406 are bumps, having a generally rounded cross-sectional contour and configured to engage a non-metal rod.

The ring body 400 further includes a first end 411 and a second end 412. Generally, the distance between the first end 411 and the second end 412 is referred to as the axial width 413 of the ring body 400. In accordance with one embodiment, the ring body 400 has an axial width 413 that is equal to or less than twice the circumference of the body 400. In certain other embodiments, the rings provided herein typically have an axial width 413 that is not greater than about 20 mm. In accordance with other embodiments, the axial width 413 can be less, such as not greater than about 9 mm, such as not greater than about 8 mm, or even not greater than about 7 mm. Still, in another embodiment, the axial width 413 of the ring body 400 is at least about 2 mm. In other embodiments, the axial width 413 is at least about 3 mm, such as at least about 4 mm, or even at least about 5 mm. In one particular embodiment, the axial width 413 of the ring body 400 is within a range between about 5 mm and about 10 mm.

The ring body 400 further includes a split 415 extending axially through the inner surface and outer surface of the ring body 400. The split 415 can facilitate the engagement of the ring body 400 on a non-metal rod. For example, during installation, the ring body 400 can be fitted around the rod, and moreover during engagement of the non-metal rod within the clamp assembly, the ring body 400 may undergo compression. The split 415 facilitates compression of the ring body 400 and thus improved engagement of the ring body 400 with a non-metal rod. Generally, the width of the split 415 is less than about 3 mm. In one embodiment, the width of the split 415 is less than about 2 mm, such as less than about 1.5 mm. In accordance with another embodiment, the width 415 is greater than about 0.1 mm, such as greater than about 0.25 mm, or even greater than about 0.5 mm. In one particular embodiment, the width of the split 415 is within a range between about 0.5 mm and about 1.5 mm.

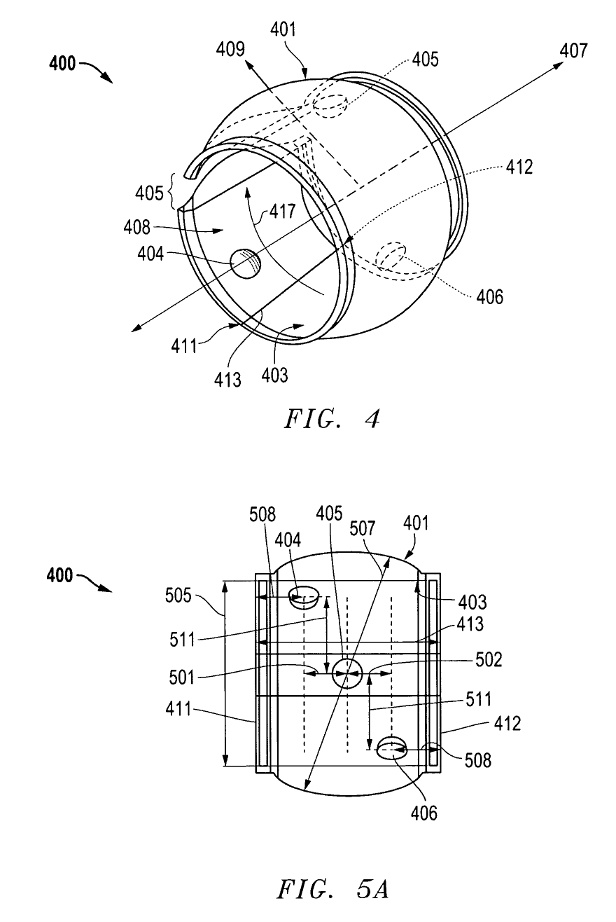

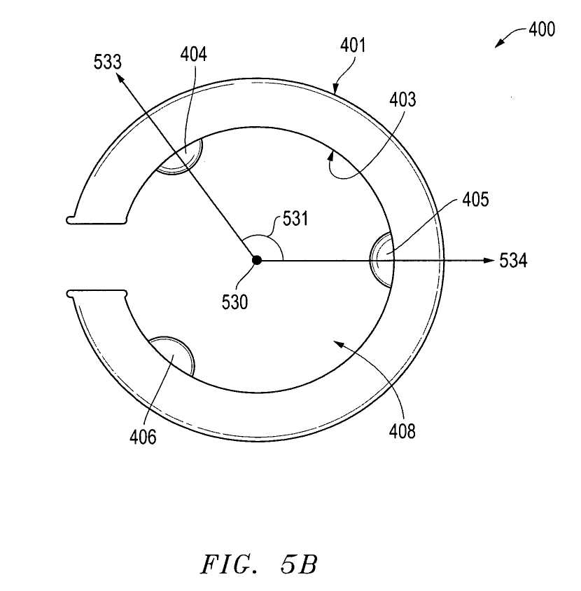

Referring to FIG. 5A, a cross-sectional view of the ring body provided in FIG. 4 is illustrated. FIG. 5A more clearly illustrates the spacing of the protrusions 404-406 along the inner surface, particularly that the protrusions 404-406 are axially spaced apart from each other. As illustrated, the protrusion 404 is axially spaced apart from protrusion 405 by a distance 501 (measured center-to-center between the protrusions) and the protrusion 405 is axially spaced apart from the protrusion 406 by a distance 502 (measured center-to-center between the protrusions). In accordance with one embodiment, the axial displacement between the protrusions 404-406 can be the same distance, such that distance 501 is equal to distance 502. Alternatively, in other embodiments, the distances 501 and 502 can be different.

In one particular embodiment, the protrusions 404-406 are axially spaced apart by at least about 5% of the axial width 413 of the ring body 400. For example, distance 501 or distance 502 is at least about 5% of the axial width 413. In another embodiment, the protrusions 404-406 are axially spaced apart by at least 10%, such as at least about 15%, or even at least about 20% of the axial width 413. Still, in another embodiment, the protrusions 404-406 are axially spaced apart by not greater than about 90% of the axial width 413 of the body. In another embodiment, the protrusions 404-406 are axially spaced apart by not greater than about 80%, such as not greater than about 70%, not greater than about 60%, or even not greater than about 50% of the axial width 413 of the ring body. In a more particular embodiment, the axial spacing between protrusions 404-406 is within a range between about 20% and about 50% of the axial width 413 of the ring body.

Moreover, the protrusions can be positioned on the inner surface 403 at a certain distance from the closest end. As illustrated in FIG. 5A, protrusions 404 and 406 are closest to the ends 411 and 412 respectively and can be positioned on the inner surface 403 such that they are a particular distance 508 away from their respective closest ends 411 and 412. As such, in accordance with the illustrated embodiment, the distance 508 is at least about 5% of the axial width 413. In other embodiments, the protrusions 404 and 406 are closer to the center of the body, such that the distance 508 is at least about 10% of the axial width, such as at least about 15% of the axial width, or at least about 20% of the axial width. Typically, for such embodiments as illustrated in FIG. 5A, to maintain suitable axial spacing between the protrusions 404-406, the distance 508 is not greater than about 40% of the axial width 413.

The circumferential spacing 511 between adjacent protrusions that are closest to each other by a circumferential measurement only (measured center-to-center between the protrusions), can depend upon the number of protrusions along the inner surface 403. However, typically in embodiments as illustrated in FIGS. 3-6C, the inner surface 403 includes at least two discrete protrusions, if not more. Accordingly, in one embodiment, the circumferential spacing 511 between discrete adjacent protrusions is generally not greater than about 50% of the total circumference of the inner surface 403. In one embodiment, the spacing is less, such as not greater than about 40% or not greater than about 30% of the total circumference of the inner surface 403. In one particular embodiment, the circumferential spacing 511 between discrete adjacent protrusions is within a range between about 5% and about 30% of the total circumference of the inner surface 403.

Referring briefly to FIG. 5B, a cross-sectional illustration of the ring body 400 is illustrated according to one embodiment. FIG. 5B more clearly illustrates that the circumferential spacing between discrete adjacent protrusions can be measured by an angle 531. For example, an angle 531 between radii 533 and 534 extending through adjacent protrusions 404 and 405 respectively from a center point 530 of the body within the aperture 408. As such, generally, the angle 531 is not greater than about 180°. In other embodiments, the angle 531 is less, such as not greater than about 160°, or not greater than about 120°. Generally, the angle 531 is at least about 10°, such as at least about 30°, or even at least 60°. According to one particular embodiment, the angle 531 between discrete adjacent protrusions 404 and 405 is within a range between about 30° and about 120°.

Referring again to FIG. 5A, in certain embodiments, the circumference of the inner surface 403 (including the distance of the split) is not greater than about 50 mm. In other embodiments, it can be less, such as not greater than about 40 mm, not greater than about 30 mm, or even not greater than about 20 mm. Typically, in accordance with an embodiment, the circumference of the inner surface 403 is at least about 5 mm. Accordingly, in other certain embodiments, the circumference is at least about 7 mm, such as at least about 10 mm, or at least about 12 mm. In one particular embodiment, the circumference of the inner surface 403 is within a range between about 12 mm and about 50 mm, and more particularly within a range between about 12 mm and about 30 mm.

As provided in FIG. 5A, the ring body includes an average inner diameter 505 measured between inner surfaces 403 across the center of the aperture. Generally, the average inner diameter of the ring body is not greater than about 15 mm. In one embodiment, the average inner diameter 505 is less, such as not greater about 12 mm, or not greater than about 10 mm. Generally, however, the average inner diameter 505 is at least about 1 mm. In a more particular embodiment, the average inner diameter 505 is at least about 3 mm, such as at least about 4 mm. In a particular embodiment, the average inner diameter 505 is within a range between about 4 mm and about 12 mm.

The ring body further includes an average outer diameter 507 measured as an average distance between points along the outer surface 401 and across the center of the ring body. In one embodiment, the average outer diameter 507 is not greater than about 20 mm. In another embodiment, the average outer diameter 507 is less, such as not greater than about 15 mm, or not greater than about 12 mm. In accordance with another embodiment, the average outer diameter 507 is generally at least about 4 mm. In another embodiment, the average outer diameter 507 is at least about 5 mm, such as at least about 6 mm. In one particular embodiment, the average outer diameter 507 is within a range between about 6 mm and about 15 mm.

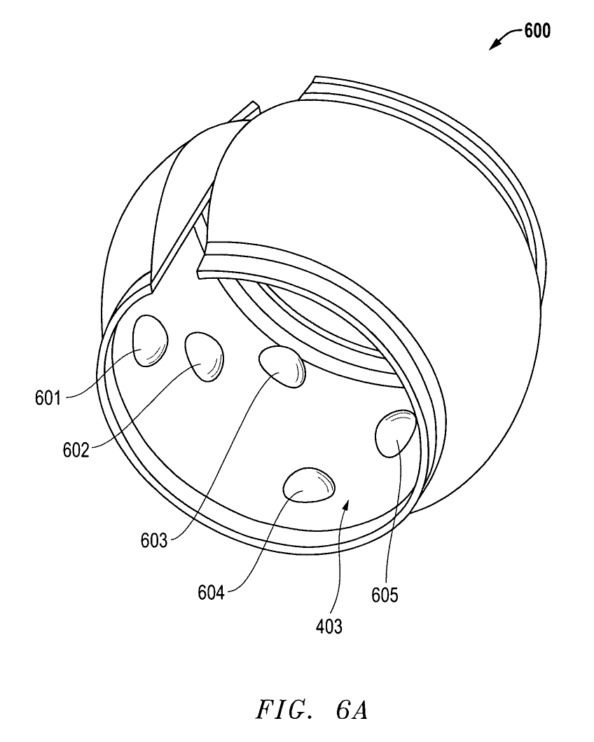

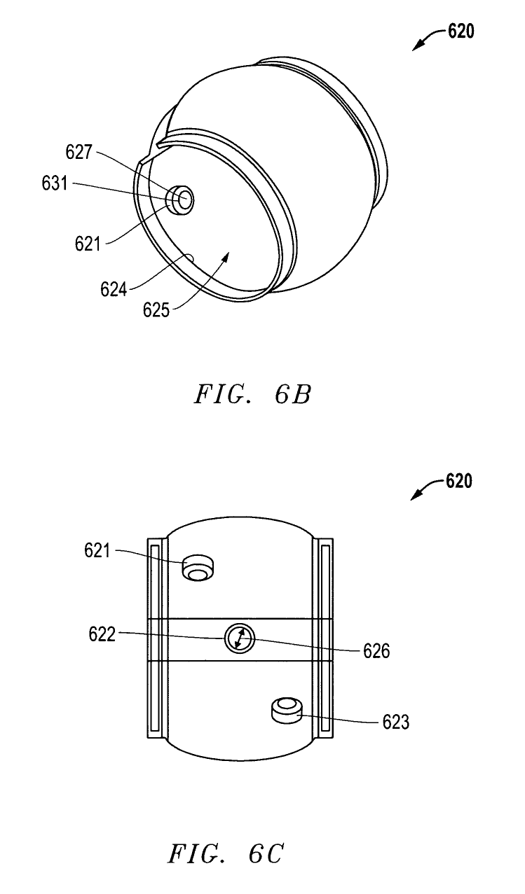

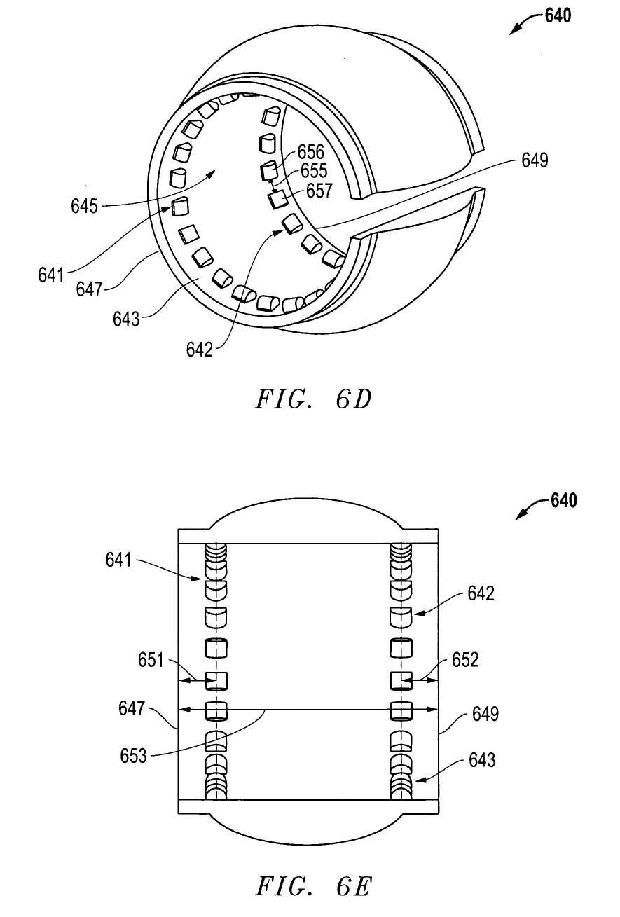

FIG. 6A includes a perspective illustration of a ring configured for use in a clamp assembly in accordance with one embodiment. In particular, the ring body 600 includes protrusions 601, 602, 603, 604, and 605 (601-605) that are axially spaced apart and circumferentially spaced apart from each other. The ring body 600 includes a greater number of protrusions 601-605. In accordance with one embodiment, the protrusion 601-605 can be provided on the inner surface 403 in a pattern, such that the axial and circumferential spacing is a regular and repeating distance. In accordance with another particular embodiment, protrusions 601-605 can be provided on the inner surface 403 of ring body 600 in a random manner, such that the spacing between the protrusions 601-605 is irregular.

According to one embodiment, the protrusions 601-605 can overly at least about 5% of the surface area of the inner surface 403. According to another embodiment, the protrusion 601-605 overly at least about 10%, such as at least about 15%, at least about 20%, or even at least about 25% of the surface area of the inner surface 403. Still, according to particular embodiments utilizing the discrete hemispherical projections illustrated in FIG. 6A, the protrusions 601-605 generally overly not greater than about 90% of the surface area of the inner surface 403. In other embodiments having discrete hemispherical projections, the protrusions 601-605 overly not greater than about 80%, such as not greater than about 70%, or even not greater than about 60% of the surface area of the inner surface 403. In one particular embodiment, the protrusion 601-605 overly at least about 25% and not greater than about 50% of the surface area of the inner surface 403.

FIG. 6B includes a perspective illustration of a ring configured for use in a clamp assembly in accordance with one embodiment. In particular, the ring body 620 includes a protrusion 621 (others illustrated in FIG. 6C) extending from the inner surface 624 into the aperture 625. According to the illustrated embodiment of FIG. 6B, the protrusion 621 has a generally cylindrical shape, including a circular cross-sectional contour and extending for a height into the aperture 625. Moreover, in a particular embodiment, the protrusion 621 can have a tapered edge 631 around the top surface 627 to avoid sharp corners which otherwise may cause localized stresses to a non-metal rod engaged by the protrusion 621. In other embodiments, the top surface 628 of the protrusion 621 can be roughened to facilitate additional slip resistance when engaged with a non-metal rod. It will be appreciated that the protrusions 621, while illustrated as having a cylindrical shape, can have other geometric shapes, such as rectangular, square, or trapezoidal.

Generally, the height of such cylindrically-shaped protrusion 621, measured as the distance between the top surface 627 and the inner surface 624 of the ring body 620, is at least about 0.25 mm. In other embodiments, the height is greater, such as at least about 0.5 mm, or at least about 1 mm. In one particular embodiment, the height of the cylindrically-shaped protrusion 621 is not greater than about 3 mm.

Referring to FIG. 6C, a cross-sectional illustration of the ring of FIG. 6B is provided. FIG. 6C more clearly illustrates that the ring body 620 includes a plurality of protrusions, 621, 622, and 623 extending from the inner surface into the aperture and spaced apart from each other axially and circumferentially along the inner surface. Like previously described embodiments, the protrusion 621-623 can be provided on the inner surface in a pattern, such that the axial and circumferential spacing is a regular and repeating distance. In accordance with another particular embodiment, protrusions 621-623 can be provided on the inner surface of ring body 620 in a random manner, such that the spacing between the protrusions 6231-623 is irregular.

The cylindrically-shaped protrusions 621-623 can have a diameter 626 measured between the sides of the protrusion of at least about 0.5 mm. In other embodiments, the diameter can be greater, such as at least about 0.75 mm, at least about 1 mm or even at least about 1.5 mm. In accordance with one particular embodiment, the cylindrically-shaped protrusions 621-623 have a diameter 626 within a range between about 0.5 mm and about 3 mm and more particularly within a range between about 0.5 mm and about 2 mm.

FIG. 6D includes a perspective illustration of a ring configured for use in a clamp assembly in accordance with one embodiment. In particular, the ring body 640 includes rows of protrusions 641 and 642 extending from the inner surface 643 into the aperture 645 of the ring body adjacent to the ends 647 and 649 respectively. As illustrated, the rows of protrusions 641 and 642 each comprise a plurality of protrusions having a semi-cylindrical shape. Notably, the upper surfaces of the protrusions within each of the rows of protrusions 641 and 642 have rounded surfaces configured to engage a non-metal rod, to reduce localized stresses on the rod that would otherwise be caused by sharp corners. It will be appreciated, that other shapes may be used, for example according to one alternative embodiment, the rows of protrusions 641 and 642 include protrusion having a hemispherical shape.

Generally, for such embodiments using rows of protrusions 641 and 642, the circumferential spacing between the individual protrusions, for example circumferential spacing 655 between protrusions 656 and 657, is at least about 1.5 mm. In another embodiment, the circumferential spacing 655 can be greater, such as at least about 3 mm, such as at least about 4 mm, or even at least about 5 mm. In part, the distance of the circumferential spacing depends upon the number of protrusions provided in the row, however, in accordance with one embodiment, the circumferential spacing 655 is not greater than about 20 mm, and more particularly not greater than about 16 mm.

In accordance with another embodiment, the rows of protrusions 641 and 642 can extend for the entire circumference of the inner surface 643. In other embodiments, the rows of protrusions 641 and 642 can extend for a fraction of the circumference of the inner surface 643. For example, in one particular embodiment, the rows of protrusions 641 and 642 can extend for a length of at least about 30% of the circumference of the inner surface 643. In other embodiments, this fraction can be greater, such as at least about 50%, such as at least about 75%, or even at least 80% of the circumference of the inner surface 643.

Moreover, in a more particular embodiment, the rows of protrusions 641 and 642 can be staggered, such that one row extends for a distance along a portion of the circumference of the inner surface 643 and terminates, and then another row of protrusions extends for a distance along a portion of the circumference of the inner surface 643. In such embodiments, the rows of protrusions are axially and circumferentially spaced apart from each other, such that one row begins and terminates before another row starts that is axially and circumferentially displaced along a portion of the inner surface 643 from the other row.

Referring to FIG. 6E, a cross-sectional illustration of the ring of FIG. 6D for use in a clamp assembly is provided in accordance with one embodiment. As illustrated, the row of protrusions 641 is spaced a distance 651 along the inner surface 643 from the closest end 647 of the ring body 640, and the row of protrusions 642 is placed a distance 652 along the inner surface 643 from the closest end 649. In accordance with an embodiment, the spacing distances 651 and 652 can be the same relative to each other. In a more particular embodiment, the spacing distances 651 and 652 are a fraction of the axial width 653 of the ring body 640 measured between the ends 647 and 649. As such, in one embodiment, the spacing distances 651 and 652 are not greater than about 30% of the axial width 653. In another embodiment, the spacing distances 651 and 652 are not greater than about 25%, or even not greater than about 20% of the axial width 653. Still, in accordance with a particular embodiment, the spacing distances 651 and 652 are at least about 5% of the axial width 653 and generally within a range between about 10% and about 25% of the axial width 653. The ring body 640 generally has an axial width 653 that is the same as the other ring bodies, such as those described in accordance with FIG. 4.

FIG. 7 includes a perspective view of a ring configured for use in a clamp assembly according to an alternative embodiment. Notably, the ring body 700 includes an inner surface 701 substantially covered by an array of protrusions. According to one particular embodiment, the inner surface 701 comprises a knurled pattern comprising an array of protrusions covering the entire inner surface 701 of the ring body 700. In a more particular embodiment, the inner surface 701 comprises a knurled pattern including an array of pyramidal shaped protrusions. Unlike embodiments provided in FIGS. 4-6, the entire inner surface 701 of the ring body 700 includes the knurled pattern. Still, like previous embodiments, the knurling can be provided in discrete locations along the inner surface 701. Accordingly, in one embodiment, the knurled surface covers not greater than about 90% of the surface area of the inner surface 701. In another embodiment, the knurled surface covers not greater than about 80%, such as not greater than about 70%, or even not greater than about 50% of the surface area of the inner surface 701. Generally, at least a portion of the inner surface 701 comprises the knurled surface, such that at least about 25% of the inner surface 701 comprises the knurled surface.

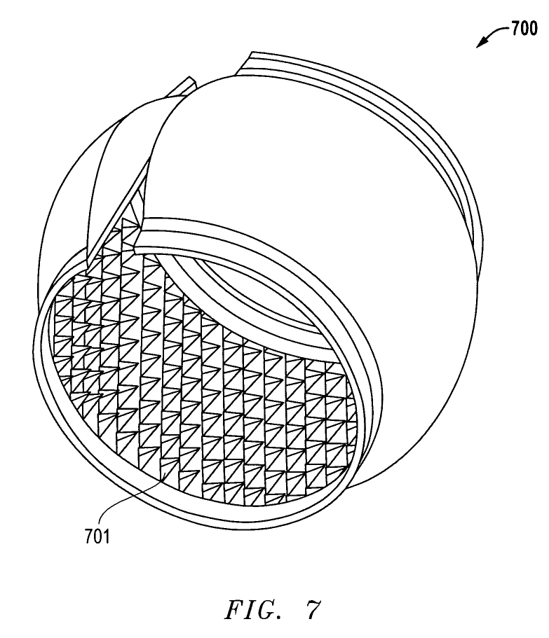

FIG. 8 includes a perspective view of a ring configured to be used in a clamp assembly in accordance with another embodiment. According to this particular embodiment, the ring body 800 includes an outer surface 801, an inner surface 803 defining an aperture 807 extending through the body 800 and configured to engage a non-metal rod and a protrusion 805 extending circumferentially along a portion of the circumference of the inner surface 803 of the body 800. Unlike previous embodiments illustrating protrusions that were generally hemispherical protrusions resembling bumps, the protrusion 805 is a ridge having a generally rectangular cross-sectional contour defining a width and a height.

Generally, the protrusion 805, can extend for at least a portion of the circumference of the inner surface 803. According to one embodiment, the protrusion 805 has a length that extends for at least about 5% of the circumference of the inner surface 803. In another embodiment, the protrusion 805 has a length that extends for at least about 10%, such as at least about 20%, or even at least 50% of the circumference of the inner surface 803. In a more particular embodiment, the protrusion 805 has a length that extends for the entire circumference of the inner surface 803. It will be appreciated, that given the split 809 present within the ring body 800 the inner surface 803 may not be a full 360°, however, it is still referred to as a circumference.

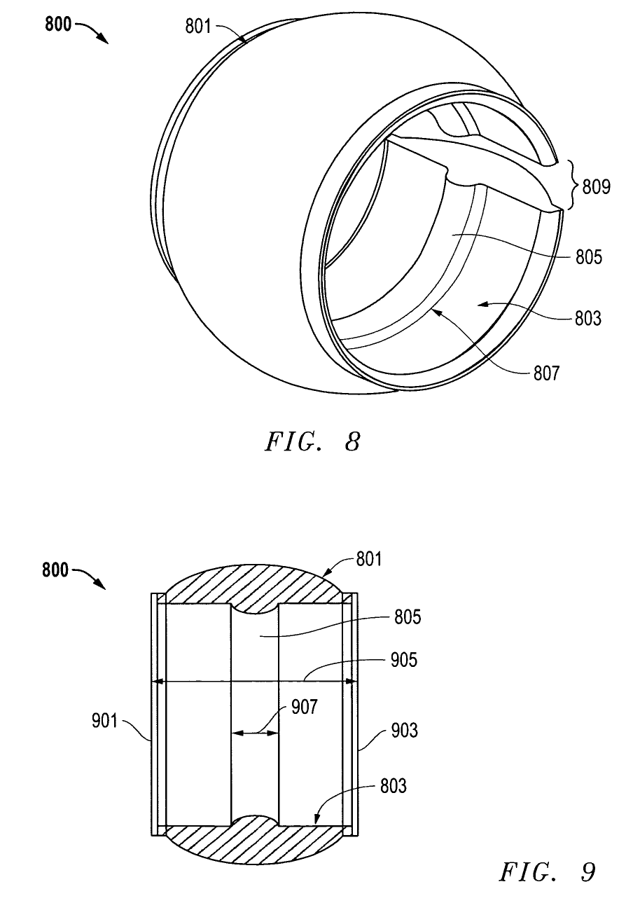

Referring to FIG. 9, a cross-sectional view of a portion of the ring body of FIG. 8 is illustrated in accordance with one embodiment. As provided in FIG. 9, the ring body 800 includes an axial width 905 defined between a first end 901 and a second end 903 of the ring body 800. According to one embodiment, the protrusion 805, or ridge, has a width 907 that is a fraction of the axial width 905. In one particular embodiment, the protrusion 805 has a width 907 that is not greater than about 75% of the axial width 905. In another more particular embodiment, the protrusion 805 has a width 907 that is not greater than about 60%, such as not greater than about 50%, such as not greater than about 30% of the axial width 905. In one particular embodiment, the protrusion 805 has a width 907 that is within a range between about 5% and about 30% of the axial width 905. It will be appreciated that the embodiment of FIGS. 8 and 9 illustrating a protrusion in the form of a ridge can be combined with other embodiments having protrusions of different contours, such as the discrete hemispherical projections, or bumps.

FIG. 10 includes a perspective view of a ring body configured to be used in a clamp assembly in accordance with another embodiment. As illustrated, the ring body 1000 includes protrusions 1001, 1002, and 1003 (1001-1003), or ridges, that extend from the inner surface 1005 and have lengths that extend circumferentially along the inner surface 1005. According to one embodiment, the protrusions 1001-1003 are ridges, having lengths the same as the embodiment described in accordance with FIG. 8. Moreover, the protrusions 1001-1003 can be substantially parallel to each other and axial displaced from each other along the inner surface 1005 by a regular spacing distance. According to one particular embodiment, while FIG. 10 illustrates the protrusions 1001 through 1003 extending parallel to each other, in another embodiment, the protrusions 1001-1003 may be staggered, such that a first protrusion extends for a length along the inner surface 1005 and terminates while an adjacent protrusion begins that is axially and circumferentially spaced apart from the first protrusion and extends along the inner surface 1005 for a length.

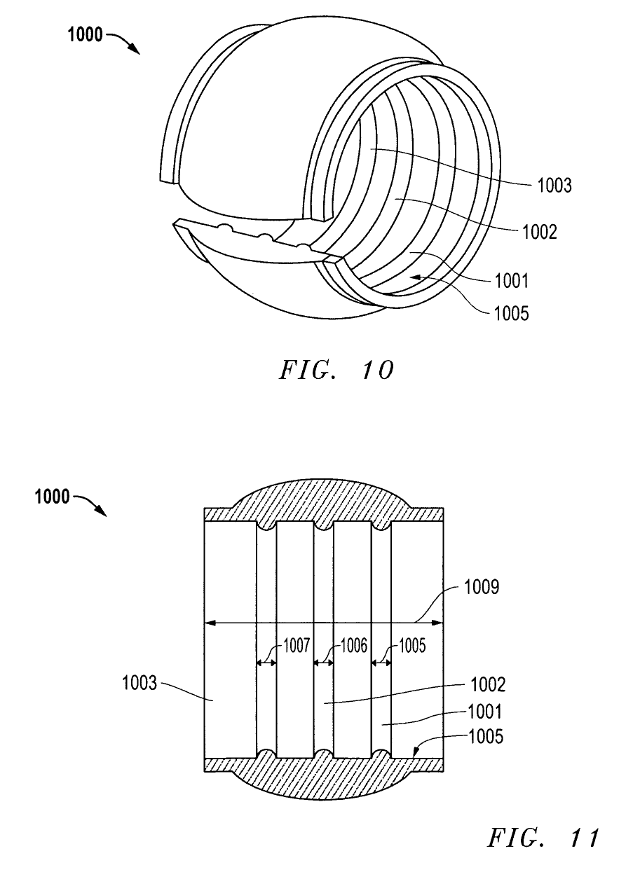

FIG. 11 provides a cross-sectional illustration of the ring body of FIG. 10 in accordance with one embodiment. As illustrates, the protrusions 1001-1003 extend along the inner surface 1005 circumferentially. Moreover, the protrusions 1001-1003 can have widths 1005, 1006, and 1007, respectively, which correspond to those widths previously described in accordance with FIG. 9. That is, the protrusions 1001-1003 typically have widths 1005-1007, respectively, which are a fraction of the axial width 1009 of the ring body 1000. Again, like the other embodiments, the protrusions illustrated in FIG. 10 can be combined with other protrusions described herein.

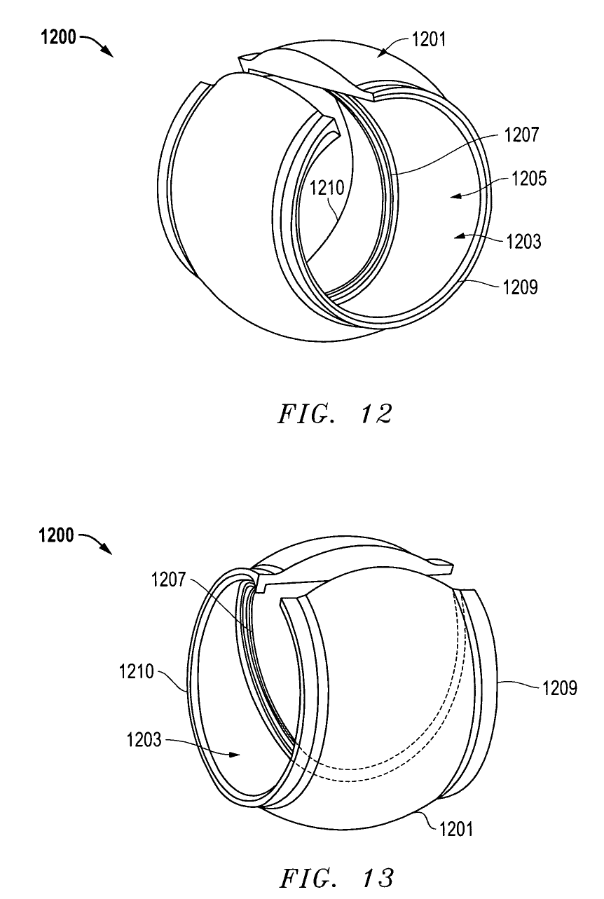

FIG. 12 includes a prospective view of a ring body configured to be used in a clamp assembly in accordance with one embodiment. As illustrated, the ring body 1200 includes an outer surface 1201, an inner surface 1203 defining an aperture 1205 extending through the body 1200, and a protrusion 1207 extending along the inner surface 1203 into the aperture 1205. As illustrated, the protrusion 1207, which can be a ridge, extends along the inner surface 1203 in a generally helical path, such that the protrusion 1207 travels in a circumferential direction along the inner surface 1203 and also in an axial direction. The protrusion 1207 can have a length the same as the protrusions described in accordance with FIG. 8. Moreover, in one particular embodiment, the protrusion 1207 can be segmented such that it is made up of multiple smaller segments, each of the smaller segments traveling in the helical path. Moreover, in another embodiment, the ring body 1200 can include a plurality of protrusions that are staggered, such that the plurality of protrusions are axially displaced and disconnected from each other. Alternatively, in another embodiment, the ring body 1200 can include a plurality of protrusions, wherein each protrusion extends in a helical path along the inner surface 1203.

FIG. 13 is a illustration of the ring body of FIG. 12 in accordance with one embodiment. Notably, FIG. 13 more clearly illustrates the protrusion 1207, or ridge, extending circumferentially along the inner surface 1203 and moreover, being axially displaced between the first end 1209 and the second end 1210, such that the protrusion 1207 has a helical path along the inner surface 1203. Like the other embodiments, the protrusions illustrated in FIGS. 12 and 13 can be combined with other protrusions described herein.

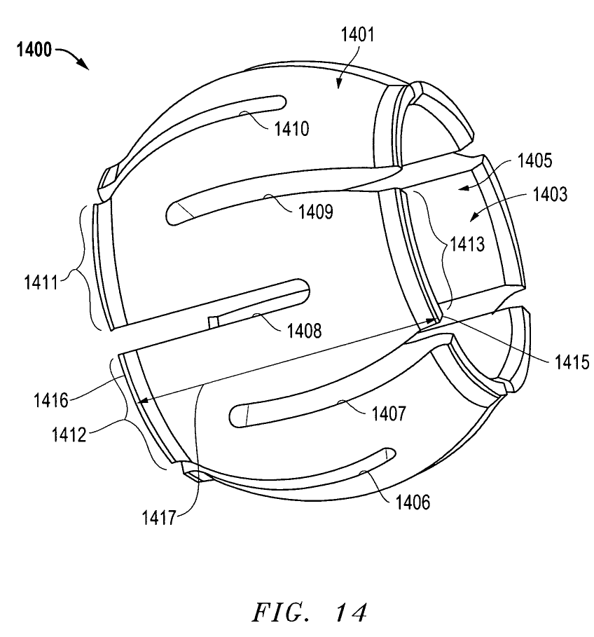

FIG. 14 includes a perspective view of a ring body configured to be used in a clamp assembly and engage a non-metal rod in accordance with one embodiment. As illustrated, the ring body 1400 includes an outer surface 1401, an inner surface 1403 defining an aperture 1405 extending through the body 1400 to engage a non-metal rod, and channels 1406, 1407, 1408, 1409 and 1410 (1406-1410) that extend axially into the body 1400 from either the first end 1415 or the second end 1416. In particular, the channels 1406-1410 can be staggered, for example, channel 1408 extends axially into the body 1400 from the second end 1416, while the two closest circumferentially adjacent channels 1407 and 1409 extend axially into the body 1400 from the first end 1415. Such a design facilitates the formation of flanges 1411, 1412 and 1413 within the body 1400. Moreover, such a design facilitates compressibility of the ring body 1400 such that a greater amount of the inner surface 1403 can be in contact with non-metal rod during implantation, thereby reducing slippage and localized stresses on the non-metal rod.

The ring body 1400 has an axial width 1417 defined as the distance between the first end 1415 and the second end 1416. Accordingly, in one embodiment the channels 1406-1410 can extend axially into the ring body 1400 for a fraction of the axial width 1417. For example, in one embodiment, the channels 1406-1410 extend axially into the ring body 1400 for a length of not greater than about 80% of the axial width 1417. In another particular embodiment, the length of the channels 1406-1410 can be less, such as not greater than about 70% of the axial width, or not greater than about 60%, or not greater than about 50%, or even not greater than about 40% of the axial width. In a more particular embodiment, the channels 1406-1410 have a length within a range between about 10% and about 80% of the axial width of the ring body 1400.

Generally, the channels 1406-1410 are spaced apart by a distance sufficient to form the flanges 1411-1413, which is sufficient to apply a suitable gripping force to a non-metal rod. Typically, in particular embodiments, the ring body 1400 has at least three channels, and more typically, at least 6 channels. Accordingly, in one embodiment, each channel is separated from a closest adjacent channel by a distance that can be measured in degrees based upon an angle between radii extending through the channels from a center point within the aperture 1405 of the body 1400. As such, in one embodiment, closest adjacent channels are separate by a distance of at least about 5°. In another more particular embodiment, closest adjacent channels are separated by at least about 10°, such as at least about 30°, or even at least about 60°. Still, according to one particular embodiment, adjacent channels are separated by not greater than about 120°. Moreover, it will be appreciated that such a design can be combined with other embodiments to include protrusions along the inner surface 1403.

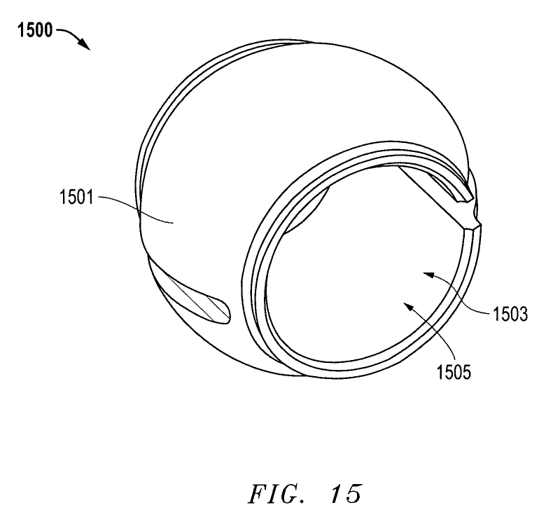

FIG. 15 includes a perspective view of a ring configured to be used in a clamp assembly in accordance with one embodiment. According to this embodiment, the ring body 1500 includes an outer surface 1501 and an inner surface 1503 defining an aperture extending through the body 1500 and configured to engage a non-metal rod. In particular, the ring body 1500 is made of a material having a Modulus of Elasticity (MOE) that is particularly matched to the MOE of the material of the non-metal rod it is configured to engage, that is, there is a maximum difference in the MOE between the material of the non-metal rod and the material of the ring body. According to one particular embodiment, the difference between the MOE of the non-metal rod and the MOE of the ring body 1500 is not greater than about 100 GPa. In other embodiments, the difference between the MOE of the non-metal rod and the MOE of the body is less, such as not greater than about 75 GPa, not greater than about 50 GPa, not greater than about 25 GPa, or even not greater than about 15 GPa. According to certain embodiments, the MOE of the material of the non-metal rod and the MOE of the material of the ring body 1500 is within a particular range, such as within a range between about 1 GPa to about 75 GPa, or more particularly between about 3 GPa and about 30 GPa, or still more particularly between 5 GPa and about 15 GPa. In another embodiment, the MOE of the material of the non-metal rod and the MOE of the material of the ring body 1500 are essentially the same. Such matching of the MOE between the material of the ring body 1500 and the non-metal rod facilitates a load sharing design that reduces localized stress to the non-metal rod.

As described herein, the ring body 1500 is configured to be engaged within an opening of a clamp assembly. In one particular embodiment, the clamp material has a MOE that is greater than the MOE of the non-metal rod and the MOE of the ring body 1500. In a more particular embodiment, the MOE of the ring body 1500 is greater than the MOE of the non-metal rod and less than the MOE of the clamp. Such embodiments also facilitate a load sharing design that avoids fatigue and possible fracture of the non-metal rod.

In reference to particular values of MOE for the ring body, in one particular embodiment, the ring body 1500 is made of a material having a MOE of not greater than about 100 GPa. In another embodiment, the ring body 1500 is made of a material having a MOE of not greater than about 90 GPa, such as not greater than about 80 GPa, not greater than about 70 GPa, not greater than about 60 GPa, not greater than about 50 GPa, or even not greater than about 30 GPa. In certain other embodiments, the ring body 1500 is made of a material having an MOE of at least about 1 GPa, such as at least about 2 GPa, or even at least about 3 GPa. In certain embodiments, the ring body 1500 is made of a material having an MOE within a range between about 1 GPa and 75 GPa, and more particularly, within a range between about 3 GPa and about 30 GPa.

While the MOE value and the proceeding description of materials are done in conjunction with FIG. 15, it should be noted that all such embodiments here can incorporate the materials and physical characteristics (e.g., MOE) of the ring body 1500. As such, generally, the ring body 1500 can include a non-metal material. Additionally, the ring body 1500 can be made of an autoclavable material, that is, a material capable of withstanding high pressures and temperatures used in autoclaving to sterilize objects. In one embodiment, suitable non-metal materials can include organics, and more particularly, polymers. Suitable polymers can include biocompatible polymers. In a more particular embodiment, suitable polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. In one particular embodiment, the ring body 1500 is made entirely of PEEK.

In certain embodiments, the polymer materials of the ring body 1500 (as well as other ring bodies or portions of ring bodies disclosed herein) can be reinforced with a filler material. Suitable filler materials can include carbon-containing materials, oxides, borides, nitrides, or any combination thereof. In one particular embodiment, the ring body 1500 is made entirely of carbon-fiber-reinforced PEEK. While the illustrated embodiment of the FIG. 15 does not illustrate protrusions, it will be appreciated that such a design can further include protrusions along the inner surface 1503 as previously presented in embodiments herein. Moreover, in an alternative embodiment, the inner surface 1503 defines and aperture 1505 having a non-circular cross-sectional contour, such that the cross-sectional contour can be elliptical, oval or oblong.

Moreover, with respect to embodiments using protrusions extending from the inner surface of the ring body into the aperture, such as those illustrated in FIGS. 3-13, the protrusions can include an autoclavable material. In fact, the protrusions can be formed of the same material as the ring body and can be fixably attached or formed as an integrated part of the ring body. As such, in one embodiment, the protrusions of the ring bodies can include a non-metal material, such as a polymer as disclosed herein. In accordance with a particular embodiment, the protrusions can include a polyether material, and more particularly PEEK. Additionally, in such embodiments utilizing a non-metal material within the protrusions, the material can include a filler material as described herein. Still, in accordance with an alternative embodiment, some ring bodies may utilize a protrusion that includes a metal or metal alloy as described herein.

FIG. 16 includes a perspective view of a ring configured for use in a clamp assembly according to one embodiment. As illustrated, FIG. 16 provides a ring body 1600 having multiple and separate portions 1601, 1602, and 1603. In a particular embodiment, each of the portions 1601-1603 can include a different material. However, in a more particular embodiment, portions 1601 and 1602 include the same material, while portion 1603 is made of a different material than portions 1601 and 1602. For example, according to one embodiment, portions 1601 and 1602 can be made of a non-metal material, while portion 1603 can be made of a metal material. Suitable non-metal materials can include those described herein, including for example, suitable polymer materials such as those materials previously described in accordance with FIG. 15. As such, in one particular embodiment, portions 1601 and 1602 include a ketone material, such as PEEK. In a more particular embodiment, portions 1601 and 1602 include a carbon reinforced PEEK.

Generally, suitable metal materials can include transitional metals. In a more particular embodiment, the portion 1603 can include a metal material such as chromium, cobalt, nickel, titanium, tungsten, aluminum, molybdenum, vanadium, or any combination thereof.

As such, the portions 1601 and 1602 can be made of a material having a MOE that is different than the MOE of the material of portion 1603. In one embodiment, the portions 1601 and 1602 can be made of a material having a MOE that is less than the MOE of the material of portion 1603. According to a particular embodiment, the portions 1601 and 1602 are made of a material having a MOE that is at least 5% less than the MOE of the portion 1603. Still, in other embodiments, the difference can be greater, such that portions 1601 and 1602 are made of a material having a MOE that is at least 10% less, such as at least 20% less, or even at least about 30% less than the MOE of the portion 1603. Generally, the difference in MOE between the material of the portions 1601 and 1602 and the material of the portion 1603 is not greater than about 90% of the MOE of portion 1603, or even more particularly, not greater than about 80%, not greater than about 75%, or even not greater than about 70%. Accordingly, ring body 1600 facilitates a load sharing design such that stresses are more evenly distributed across the non-metal rod during engagement within the ring body 1600.

Moreover, portions 1601 and 1602 can also be suitably matched to the non-metal rod, such that the difference in MOE is not greater than 50 GPa. Such a difference in MOE between the portions 1601 and 1602 and the non-metal rod facilitate a load sharing design as previously identified.

According to another embodiment, portions 1601 and 1602 are configured to form the ends or flanges of ring body 1600. Such a design facilitates reduced stress on the non-metal rod as edge portions 1608 and 1609 of portions 1601 and 1602 respectively, have physical characteristics more closely matching the non-metal rod and thus less likely to cause localized stress to the non-metal rod.

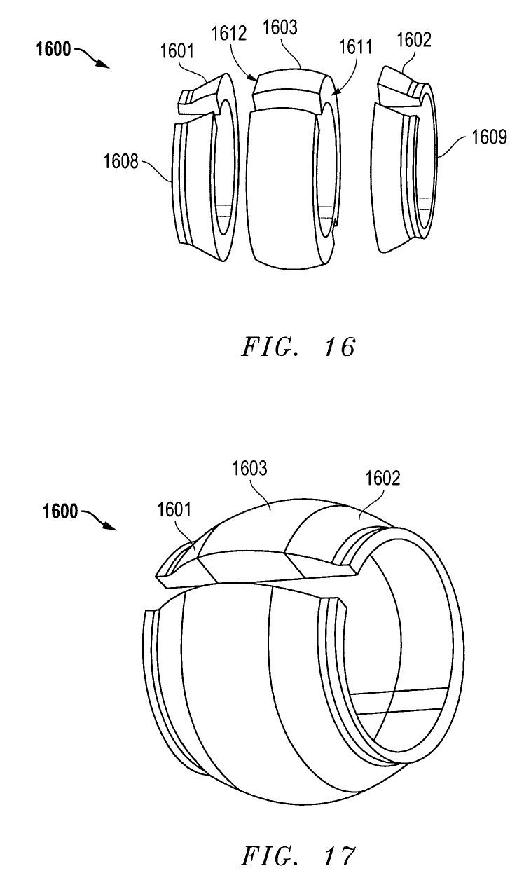

FIG. 17 includes a perspective view of the ring body 1600 as illustrated in FIG. 16 after assembly. According to one embodiment, portions 1601 and 1602 can be coupled onto portion 1603. Coupling of the portions can include an adhesive, or alternatively, a physical attachment, such as by an interference fit, molding, or the like. As such, in one embodiment, portions 1601 and 1602 can be selectively coupled or decoupled, and more particularly interchangeable with a plurality of different portions, wherein each of the portions can be made of different materials and have different physical characteristics. Such a design facilitates selection of portions 1601 and 1602 by the surgeon having physical characteristics most compatible for integration with the non-metal rod that are specifically tailored to operation of the implant.

In an alternative embodiment, portions 1601 and 1602 are fixably attached to the central portion 1603 during formation of the ring body 1600. As such, one method of forming the ring body 1600 can include injection molding of end portions 1601 and 1602 onto the central portion 1603. In a particular embodiment, the portion 1603 can include a metal material having holes displaced along its side surfaces 1611 and 1612 such that upon placement in a die for injection molding, the softer material forming portions 1601 and 1602 (e.g., a non-metal material, such as PEEK) is injected within the holes for a stronger connection between the portions 1601-1603. Moreover, protrusions can be provided on any one of the inner surfaces on any one of the portions 1601-1603 illustrated herein.

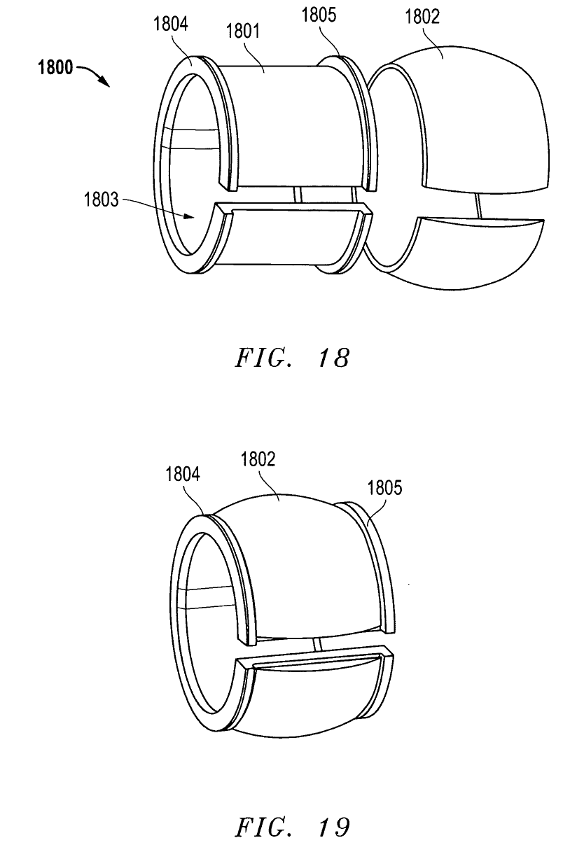

FIGS. 18 and 19 illustrate a ring configured for use with a clamp assembly and having multiple portions according to another embodiment. As illustrated in FIG. 18, the ring body 1800 includes multiple and separate portions, including an inner portion 1801 and an outer portion 1802. Like previous embodiments illustrated in FIGS. 16 and 17, the inner portion 1801 and outer portion 1802 can included different materials. As such, in one particular embodiment, the inner portion 1801 includes a non-metal material, while portion 1802 includes a metal. As such, the inner portion 1801 can include a polymer material. In one embodiment, the inner portion 1801 can include a PEEK material. According to another embodiment, the outer portion 1802 can include a metal material, such as those previously identified in accordance with FIG. 16.

Portions 1801 and 1802 can further include particular physical characteristics (e.g., MOE) with relation to each other and the non-metal rod as described in accordance with embodiments of FIGS. 16 and 17. In another more particular embodiment, inner portion 1801 includes a material having a particular MOE as compared to the MOE of the material of the non-metal rod it is configured to engage as described in accordance with other embodiments herein.

Notably, the inner surface 1803 of the inner portion 1801 is configured to be in full contact with a non-metal rod, such that the outer portion 1802 is not in direct contact with the non-metal rod. Such a design is suitable, wherein the outer portion 1802 is a more rigid material than the inner portion 1801 and thus the non-metal rod is shielded from direct contact with the more rigid material. Additionally, in one embodiment, the inner portion 1801 includes flanges 1804 and 1805, more clearly illustrated in FIG. 19, as extending beyond the ends of the outer portion 1802. Such a design further reduces direct contact between the non-metal rod and the outer portion 1802. Accordingly, like previous designs described herein, the ring body 1800 facilitates a load sharing design. Additionally, the ring body 1800 can further include protrusions such as those described in embodiments herein.

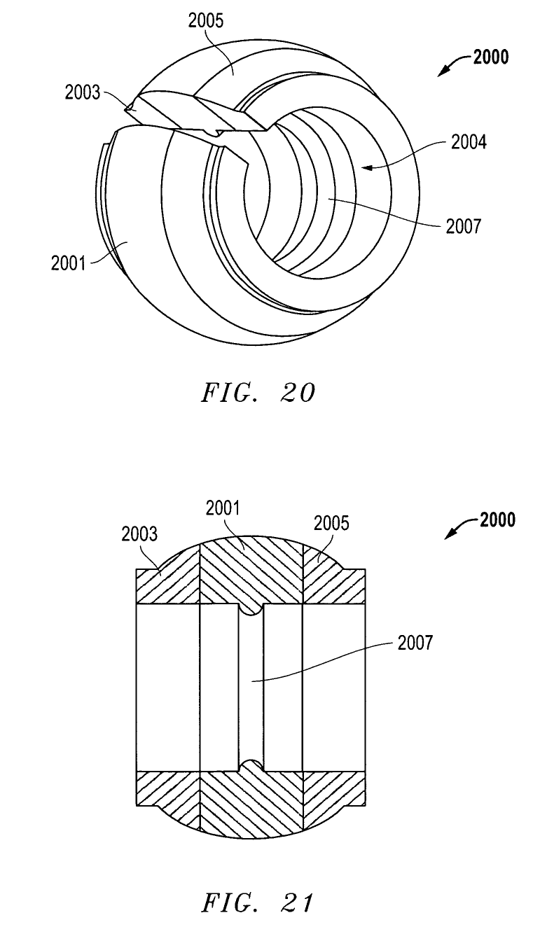

FIG. 20 includes a perspective view of a ring having multiple portions and configured for use in a clamp assembly in accordance with one embodiment. In particular, the ring body 2000 includes a portion 2001 disposed between and connected to portions 2003 and 2005 that form the ends of the ring body 2000. In accordance with one embodiment, the portion 2001 can include a material having a MOE that is different than the material comprising portions 2003 and 2005. For example, portions 2001 can include a material having a MOE that is different than an MOE of the material comprising portions 2003 and 2005 as described herein. In a more particular embodiment, the portion 2001 includes a metal or metal alloy material, while portions 2003 and 2005 include a non-metal material as described herein.

As further illustrated in FIG. 20, the ring body 2000 can further include a protrusion 2007 that is configured to engage a non-metal rod extending through the aperture 2004. In particular, the protrusion 2007 extends from the inner surface of the portion 2001. Moreover, the protrusion 2007 can be a ridge that extends for the entire circumference of the inner surface of portion 2007. Accordingly, the protrusion 2007 can have those attributes of other ring bodies using ridges as described in accordance with FIGS. 8-13. As such, in one alternative embodiment, the portion 2001 can include a protrusion 2007 in the shape of a ridge, and extending along the inner surface of the portion 2001 in a helical path.

FIG. 21 includes a cross-sectional view of the ring of FIG. 20 configured for use in a clamp assembly in accordance with one embodiment. FIG. 21 more clearly illustrates the protrusion 2007 extending from the inner surface of portion 2001 in accordance with one embodiment. In a particular embodiment, the protrusion 2007 is fixably connected to the portion 2001 and is made of the same material as portion 2007. In a more particular embodiment, the protrusion 2007 includes a metal or metal alloy material as described herein.

While not illustrated, in an alternative embodiment, the portions 2003 and 2005 can also include protrusions extending from their respective inner surfaces configured to engage a non-metal rod within the aperture. In one embodiment, protrusions extending from the portions 2003 and 2005 can be fixably connected to and be made of the same material of the portions 2003 and 2005. In a more particular embodiment, protrusions extending from the portions 2003 and 2005 can be made of a material having a MOE that is less than the MOE of the material of portion 2001 and the MOE of the material forming the protrusion 2007.

In some instances, it is the preference of the surgeon to use a non-metal rod to avoid stress shielding effects and thereby improving the bone density recovery of the patient to a more healthy state. However, the use of non-metal rods, such as PEEK rods may not be as durable as their metal counterparts, and therefore more subject to fatigue and fracture, especially when coupled with more rigid, metal components. Embodiments provided herein describe rings configured to be engaged within clamp assemblies and particularly suited for coupling non-metal rods and anchors for implantation into a human body. The ring bodies described herein provide notable improvements over the state-of-the-art, including features along the inner surface, such as protrusions, creating greater translational and rotational resistance for the rod and thereby reducing slippage of the rod in the ring. Moreover, currently disclosed embodiments disclose load-sharing designs, such as the hybrid ring designs having multiple and separate portions capable of reducing localized stress to non-metal rods within the ring. Reduction of localized stresses on the non-metal rod lessen the potential for fatigue or fracture, extending the lifetime and improving the quality of the implant.

In summary, according to a first aspect, an implantable article for use with an anchor and a non-metal rod assembly is disclosed that includes a ring having a body including an outer surface, an inner surface defining an aperture extending through the body configured to engage a non-metal rod, and protrusions extending from the inner surface into the aperture, wherein the protrusions are spaced apart from each other axially and circumferentially along the inner surface. In one embodiment of the first aspect, the body has an axial width extending between a first end of the body and a second end of the body, wherein the protrusions are axially spaced apart by at least about 5% of the axial width of the body. In a more particular embodiment, the protrusions are axially spaced apart by not greater than about 90% of the axial width of the body.

According to another embodiment of the first aspect, the body has an axial width extending between a first end of the body and a second end of the body and the body further includes channels extending axially through the body for a length less than the axial width of the body. In another embodiment, the inner surface has a surface area and the protrusions overlie at least about 5% of the surface area. Still, according to a particular embodiment, the protrusions overlie the surface area of the inner surface within a range between about 5% and about 50%.

In one embodiment of the first aspect, the non-metal rod comprises a polymer material. In a more particular embodiment, the polymer material is selected from the group of polymers consisting of polyurethane, polyolefin, polyether, silicone, or a combination thereof. In another more particular embodiment, the polymer material includes polyetheretherketone (PEEK).

According to another embodiment of the first aspect, the protrusions are discrete hemispherical projections. In a more particular embodiment, the protrusions are arranged in a pattern. In one embodiment, the protrusions include a knurled pattern on the inner surface. In another embodiment, the entire inner surface of the body comprises the knurled pattern.

Still, in another embodiment, the protrusions are ridges extending circumferentially along the inner surface. As such, in one particular embodiment, the inner surface has a circumference and the ridges have a length extending for at least about 5% of the circumference of the inner surface. In another more particular embodiment, the ridges extend for an entire circumference of the inner surface. According to another particular embodiment, the ridges extends in a helical path along the inner surface.

According to another embodiment of the first aspect, the body further comprises a split extending axially through the inner surface and outer surface. Still, in another embodiment, the body comprises a material selected from the group of materials consisting of a metal, a polymer, or any combination thereof. According to one embodiment, the material comprising the body further comprises a filler selected from the group of materials consisting of carbon, oxides, borides, nitrides, or any combination thereof. In another more particular embodiment, the body is made entirely of an autoclavable material. According to one particular embodiment of the first aspect, the inner surface defines an aperture having a non-circular cross-sectional contour.

According to another embodiment of the first aspect, the body has a Modulus of Elasticity (MOE) and the non-metal rod has a Modulus of Elasticity (MOE) and the difference between the MOE of the body and the MOE of the non-metal rod is not greater than about 50 GPa. In a more particular embodiment, wherein the body includes a material having the same MOE as the non-metal rod. In another particular embodiment, the body includes a Modulus of Elasticity (MOE) of not greater than about 100 GPa.

According to a second aspect, an implantable article for use with an anchor and non-metal rod assembly includes a ring having a body including an outer surface and an inner surface defining an aperture extending through the body configured to engage a non-metal rod. The body includes a first channel that extends axially into the body from a first end, and a second channel circumferentially displaced from the first channel along the body that extends axially into the body from a second end different than the first end. According to one embodiment, the first channel and second channel extend for a length of not greater than about 80% of the axial width. In another embodiment, the first channel and second channel extend for a length of not greater than about 60% of the axial width.

According to a third aspect, an implantable article for use with an anchor and a non-metal rod assembly includes a ring having a body including an outer surface, an inner surface defining an aperture extending through the body configured to engage a non-metal rod, and a first protrusion extending circumferentially around a portion of a circumference of the inner surface of the body. In one embodiment of the third aspect, the first protrusion extends along the entire circumference of the inner surface. As such, in a more particular embodiment, the first protrusion further extends in an axial direction along the inner surface. In another particular embodiment, the ring body further includes a second protrusion parallel to the first protrusion and extending circumferentially along the circumference of the inner surface and axially displaced from the first protrusion.

In a fourth aspect, an implantable article for use with an anchor and non-metal rod assembly includes a clamp having an opening and configured to engage an anchor and a ring configured to fit within the opening of the clamp. The ring includes a body having an outer surface, and an inner surface defining an aperture extending through the body configured to engage a non-metal rod, wherein the body comprises a Modulus of Elasticity (MOE) and the non-metal rod comprises a MOE and the difference between the MOE of the non-metal rod and the MOE of the body is not greater than about 50 GPa. In one particular embodiment of the fourth aspect, the difference between the MOE of the non-metal rod and the MOE of the body is not greater than about 40 GPa. According to another embodiment, the clamp has a MOE that is greater than the MOE of the non-metal rod and the MOE of the body. Still, in a one particular embodiment, the MOE of the body is greater than the MOE of the non-metal rod and less than the MOE of the clamp.

According to a fifth aspect, an implantable article for use with an anchor and non-metal rod assembly includes a ring having a body including an outer surface and an inner surface defining an aperture extending through the body configured to engage the non-metal rod, wherein the body comprises a first portion comprising a first material and a second portion comprising a second material different than the first material. As such, in one embodiment, the first material is metal. According to another embodiment, the second material is non-metal. Still, in a more particular embodiment, the second material includes the same material as the non-metal rod.

According to another embodiment of the fifth aspect, the body includes a first end extending circumferentially around the body and a second end extending circumferentially around the body and axially spaced apart from the first end by a width, wherein the first end and the second end include the second material. In another more particular embodiment, the body further includes a first flange attached to the first end and a second flange attached to the second end, wherein the first flange and the second flange include the second material.

In another embodiment of the fifth aspect, the outer surface comprises the first material and the second material. According to another particular embodiment, the outer surface includes only the first material and the inner surface includes only the second material.

In another embodiment of the fifth aspect, the inner surface comprises protrusions spaced apart from each other axially and circumferentially along the inner surface. Still, according to another embodiment, the first material comprises a first MOE and the second material comprises a second MOE and the second MOE is at least about 5% less than the first MOE.

The above-disclosed subject matter is to be considered illustrative, and not restrictive, and the appended claims are intended to cover all such modifications, enhancements, and other embodiments that fall within the true scope of the present invention. For example, it is noted that the components in the exemplary embodiments described herein as having a particular function or as being located in a particular housing are illustrative and it is noted that such components can perform additional functions or be located in different configurations. Thus, to the maximum extent allowed by law, the scope of the present invention is to be determined by the broadest permissible interpretation of the following claims and their equivalents, and shall not be restricted or limited by the foregoing detailed description.

What is claimed is:

1. An implantable article for use with an anchor and a non-metal rod assembly comprising:
a ring having a body including an outer surface, an inner surface defining an aperture extending through the body configured to engage a non-metal rod, and protrusions extending from the inner surface into the aperture, wherein the protrusions are spaced apart from each other axially and circumferentially along the inner surface.

2. The implantable article of claim 1, wherein the body has an axial width extending between a first end of the body and a second end of the body, wherein the protrusions are axially spaced apart by at least about 5% of the axial width of the body.

3. The implantable article of claim 1, wherein the inner surface has a surface area and the protrusions overlie at least about 5% of the surface area.

4. The implantable article of claim 1, wherein the protrusions are discrete hemispherical projections.

5. The implantable article of claim 1, wherein the protrusions are arranged in rows extending circumferentially around the inner surface of the ring body.

6. The implantable article of claim 5, wherein the ring body comprises a first end and a second end and comprises a first row of protrusions adjacent to and spaced apart from the first end along the inner surface and a second row of protrusions adjacent to and spaced apart from the second end along the inner surface.

7. The implantable article of claim 1, wherein the protrusions are ridges extending circumferentially along the inner surface.

8. The implantable article of claim 7, wherein the ridges extends in a helical path along the inner surface.

9. The implantable article of claim 1, wherein the protrusions include a knurled pattern on the inner surface.

10. The implantable article of claim 1, wherein the body comprises a material selected from the group of materials consisting of a metal, a polymer, or any combination thereof.

11. The implantable article of claim 10, wherein the body comprises polyetheretherketone (PEEK).

12. The implantable article of claim 10, wherein the material comprising the body further comprises a filler selected from the group of materials consisting of carbon, oxides, borides, nitrides, or any combination thereof.

13. An implantable article for use with an anchor and non-metal rod assembly comprising:
a ring having a body including an outer surface and an inner surface defining an aperture extending through the body configured to engage a non-metal rod, wherein a first channel extends axially into the body from a first end, and a second channel circumferentially displaced from the first channel along the body extends axially into the body from a second end different than the first end.

14. The implantable article of claim 13, wherein the body comprises an axial width defined as a distance between the first end and the second end, and the channels extend for a length of not greater than about 80% of the axial width.

15. An implantable article for use with an anchor and a non-metal rod assembly comprising:
a ring having a body including an outer surface, an inner surface defining an aperture extending through the body configured to engage a non-metal rod, and a first protrusion extending circumferentially around a portion of a circumference of the inner surface of the body.

16. The implantable article of claim 15, wherein the first protrusion further extends in an axial direction along the inner surface.

17. The implantable article of claim 15, further comprising a second protrusion parallel to the first protrusion and extending circumferentially along the circumference of the inner surface and axially displaced from the first protrusion.

18. An implantable article for use with an anchor and non-metal rod assembly comprising:
a clamp having an opening and configured to engage an anchor;
a ring configured to fit within the opening of the clamp having a body including an outer surface and an inner surface defining an aperture extending through the body configured to engage a non-metal rod, wherein the body comprises a Modulus of Elasticity (MOE) and the non-metal rod comprises a MOE and the difference between the MOE of the non-metal rod and the MOE of the body is not greater than about 100 GPa.

19. The implantable article of claim 18, wherein the clamp comprises a MOE that is greater than the MOE of the non-metal rod and the MOE of the body.

20. An implantable article for use with an anchor and non-metal rod assembly comprising:
a ring having a body including an outer surface and an inner surface defining an aperture extending through the body configured to engage the non-metal rod, wherein the body comprises a first portion comprising a first material and a second portion comprising a second material different than the first material.

21. The implantable article of claim 20, wherein the first material is metal.

22. The implantable article of claim 20, wherein the second material is non-metal.

23. The implantable article of claim 20, wherein the body comprises a first end extending circumferentially around the body and a second end extending circumferentially around the body and axially spaced apart from the first end by a width, wherein the first end and the second end comprise the second material.

24. The implantable article of claim 20, wherein the outer surface comprises the first material and the second material.

25. The implantable article of claim 20, wherein the first material comprises a first MOE and the second material comprises a second MOE and the second MOE is at least about 5% less than the first MOE.
